(19) United States
(12) Patent Application Publication
(10) Pub. No.: US 2024/0277543 A1
(43) Pub. Date: Aug. 22, 2024

(54) PATIENT SUPPORT APPARATUS WITH PATIENT WEIGHT MONITORING

(71) Applicant: Stryker Corporation, Kalamazoo, MI (US)

(72) Inventors: Sujay Sukumaran, Portage, MI (US); Madhu Thomas, London, Ontario (CA); Berkay Güncan, Talas, Kayseri (TR); Seyed Behrad Ghodsi, Portland, OR (US); Christian Fulljames, Grand Rapids, MI (US); Curt Stienstra, Lowell, MI (US); Daniel Dehoog, Grand Rapids, MI (US)

(21) Appl. No.: 18/573,228

(22) PCT Filed: Oct. 7, 2022
(86) PCT No.: PCT/US2022/046080

§ 371 Date: Dec. 21, 2023

Related U.S. Application Data
(60) Provisional application No. 63/255,223, filed on Oct. 13, 2021.
(60) Provisional application No. 63/342,902, filed on May 17, 2022.

(51) Int. Cl. A61G 7/05; G01G 19/44

(52) U.S. Cl. CPC A61G 7/0527; G01G 19/445; A61G 2203/20; A61G 2203/32; A61G 2203/44

(57) ABSTRACT

A patient support apparatus, such as a bed, cot, stretcher, etc., includes a scale system adapted to automatically monitor changes in a patient's weight over the course of a patient's stay within a healthcare facility. A controller onboard the patient support apparatus is configured to take an initial weight reading of the patient when he or she is first assigned to the patient support apparatus, and to thereafter repetitively compare the patient's current weight to that initial weight reading. If the difference exceeds a threshold, the controller issues a notification. The threshold may be user-customizable and/or it may be adaptive based on s statistical analysis of the patient's weight fluctuations. A control panel may be included to allow a caregiver to select between using a non-adaptive threshold or an adaptive threshold, and/or to select a value for the non-adaptive threshold. The notification may be transmitted to the caregiver's smart phone.

### CROSS-REFERENCE TO RELATED APPLICATIONS

This application claims priority to U.S. provisional patent application Ser. No. 63/255,223 filed Oct. 13, 2022, by inventors Sujay Sukumaran et al. and entitled PATIENT SUPPORT APPARATUS WITH PATIENT WEIGHT MONITORING, and to U.S. provisional patent application Ser. No. 63/342,902 filed May 17, 2022, by inventors Sujay Sukumaran et al. and entitled PATIENT SUPPORT APPARATUS WITH PATIENT WEIGHT MONITORING, the complete disclosures of both of which are incorporated herein by reference.

### BACKGROUND

The present disclosure relates to patient support apparatuses, such as beds, cots, stretchers, recliners, or the like. More specifically, the present disclosure relates to patient support apparatuses that include a scale system.

Existing hospital beds and/or stretchers often include a scale system that is used for weighing the patient. Many healthcare facilities require that a caregiver periodically use the scale system to take a weight reading of the patient and record that weight reading. In many instances, the bed or stretcher will provide the caregiver with an indication of how much the patient's weight has changed since the last time the patient's weight was recorded. Often, however, the bed or stretcher will not provide an indication of how much the patient's weight has changed since the patient first entered the healthcare facility, or since the patient was first assigned to the bed or stretcher. The caregiver may therefore only be presented with incremental weight change information (i.e. weight changes since the last weight was recorded), rather than cumulative weight change information (i.e. total weight change since the patient was admitted to the healthcare facility).

### SUMMARY

According to various embodiments, an improved patient support apparatus is provided that helps caregivers receive notifications regarding the cumulative weight change of a patient since he or she was first admitted to a healthcare facility, and/or since he or she was first assigned to a particular patient support apparatus. Additionally, the patient support apparatus is provided with one or more notification options regarding the patient's cumulative weight change. For example, the patient support apparatus allows the caregiver to select what criteria are used to trigger a notification regarding the patient's cumulative weight change. In some embodiments, the caregiver can specify that the notification occurs when the cumulative weight change exceeds a threshold selected by the caregiver. Alternatively, or additionally, the notification may be set to occur when the cumulative weight change exceeds a statistical value derived from measured fluctuations in the patient's weight over a period of time. Still further, in some embodiments, the caregiver can receive notifications of cumulative patient weight changes that exceed a threshold on his or her smart phone. Still other features and aspects of the present disclosure will be apparent to those skilled in the art in light of the accompanying drawing and the following written description.

A patient support apparatus according to a first aspect of the present disclosure includes a support surface, a plurality of force sensors, a memory, a control, a display, and a controller. The support surface is adapted to support a patient thereon. The plurality of force sensors are adapted to detect downward forces exerted on the support surface. The control is adapted to record a patient weight when activated by a caregiver. The controller is adapted to record in the memory an initial patient weight corresponding to a first time the caregiver uses the control to record a particular patient's weight. The controller is also adapted to thereafter automatically compare a current patient weight to the initial patient weight and to provide a notification to the caregiver if the current patient weight differs from the initial patient weight by more than a threshold. The controller is further adapted to display a threshold selection screen on the display that allows the caregiver to adjust the threshold.

According to other aspects of the present disclosure, the patient support apparatus further includes a new patient control adapted to be activated by the caregiver when a new patient is assigned to the patient support apparatus. The controller is adapted to erase the initial patient weight of the particular patient when the new patient control is activated.

The controller, in some aspects, is further adapted to store in the memory a new initial patient weight corresponding to a first time the caregiver uses the control to record a new patient's weight, and to thereafter automatically compare a current weight of the new patient to the new initial patient weight and to provide the notification to the caregiver if the current weight of the new patient differs from the new initial patient weight by more than the threshold.

The controller, in some aspects, is further adapted to display a patient weight gain/loss indicator on the display that provides an indication to the caregiver of a difference between the current patient weight and a last saved patient weight. The last saved patient weight corresponds to the weight of the patient recorded when the caregiver most recently activated the control.

The patient support apparatus, in some aspects, includes a network transceiver adapted to communicate with a server hosted on a healthcare facility computer network, and the controller is further configured to send a notification message to the server if the current patient weight differs from the initial patient weight by more than the threshold.

In some aspects, the controller is further adapted to display an adaptive threshold option on the display. If the caregiver selects the adaptive threshold option, the controller is further adapted to switch to providing the notification to the caregiver if the current patient weight differs from the initial patient weight by more than an adaptive threshold.

The controller, in some aspects, is adapted to select a value for the adaptive threshold that is based on fluctuations in the patient's weight over a time period.

In some aspects, the controller is further adapted to determine a mean absolute deviation of the fluctuations and to select the value for the adaptive threshold based on the mean absolute deviation of the fluctuations.

The controller may, in some aspects, select a value for the adaptive threshold that is greater than the mean absolute deviation.

In some aspects, the controller is adapted to increase the time period.

The threshold selection screen, in some aspects, includes a slider icon displayed thereon adapted to be repositioned by the caregiver. The user can adjust the threshold by sliding the slider icon.

The slider icon, in some aspects, is adapted to slide within a range of values that includes values between at least one and two kilograms.

According to another aspect of the present disclosure, a patient support apparatus is provided that includes a support surface, a plurality of force sensors, a control, a memory, a display, and a controller. The support surface is adapted to support a patient thereon. The plurality of force sensors are adapted to detect downward forces exerted on the support surface. The control is adapted to record a patient weight when activated by a caregiver. The controller is adapted to record in the memory an initial patient weight corresponding to a first time the caregiver uses the control to record a particular patient's weight. The controller is further adapted to thereafter automatically compare a current patient weight to the initial patient weight and to provide a notification to the caregiver if the current patient weight differs from the initial patient weight by more than an adaptive threshold. The controller is adapted to select a value for the adaptive threshold that is based on fluctuations in the patient's weight over a time period.

According to other aspects of the present disclosure, the controller is further adapted to determine a mean absolute deviation of the fluctuations and to select the value for the adaptive threshold based on the mean absolute deviation of the fluctuations.

In some aspects, the controller is further adapted to select the value for the adaptive threshold that is greater than the mean absolute deviation.

The controller may be adapted to increase the time period.

The patient support apparatus, in some aspects, includes a new patient control adapted to be activated by the caregiver when a new patient is assigned to the patient support apparatus. The controller is adapted to erase the initial patient weight of the particular patient when the new patient control is activated.

The controller is further adapted, in some aspects, to store in the memory a new initial patient weight corresponding to a first time the caregiver uses the control to record a new patient's weight, and to thereafter automatically compare a current weight of the new patient to the new initial patient weight and to provide the notification to the caregiver if the current weight of the new patient differs from the new initial patient weight by more than the adaptive threshold.

The controller, in some aspects, is further adapted to display a patient weight gain/loss indicator on the display that provides an indication to the caregiver of a difference between the current patient weight and a last saved patient weight. The last saved patient weight corresponds to the weight of the patient recorded when the caregiver most recently activated the control.

The patient support apparatus, in some aspects, also includes a network transceiver adapted to communicate with a server hosted on a healthcare facility computer network. The controller may be further configured to send a notification message to the server if the current patient weight differs from the initial patient weight by more than the adaptive threshold.

In some aspects, the controller is further adapted to perform the following: (i) during the time period, compare a current patient weight to the initial patient weight and to provide the notification to the caregiver if the current patient weight differs from the initial patient weight by more than a non-adaptive threshold; and (ii) subsequent to the time period, compare a current patient weight to the initial patient weight and to provide the notification to the caregiver if the current patient weight differs from the initial patient weight by more than the adaptive threshold.

In some aspects, the controller is further adapted to display a threshold selection screen on the display that is adapted to allow the caregiver to adjust the non-adaptive threshold.

The threshold selection screen, in some aspects, includes a slider icon displayed thereon adapted to be repositioned by the caregiver in order for the caregiver to select the non-adaptive threshold.

The slider icon, in some aspects, is adapted to slide within a range of values that includes values between at least one and two kilograms.

In some aspects, the patient support apparatus further includes a selection control in communication with the controller. The selection control is adapted to allow the caregiver to select between a first patient monitoring technique and a second patient monitoring technique, wherein the controller is further adapted to perform the following: (i) when the caregiver has selected the first patient monitoring technique, compare a current patient weight to the initial patient weight and provide the notification to the caregiver if the current patient weight differs from the initial patient weight by more than a non-adaptive threshold; and (ii) when the caregiver has selected the second patient monitoring technique, compare a current patient weight to the initial patient weight and provide the notification to the caregiver if the current patient weight differs from the initial patient weight by more than the adaptive threshold.

According to another aspect of the present disclosure, a patient support apparatus is provided that includes a support surface, a plurality of force sensors, a control, a selection control, and a memory. The support surface is adapted to support a patient thereon. The plurality of force sensors are adapted to detect downward forces exerted on the support surface. The control is adapted to record a patient weight when activated by a caregiver. The selection control is adapted to allow the caregiver to select between a first patient monitoring technique and a second patient monitoring technique. The controller is adapted to record in the memory an initial patient weight corresponding to a first time the caregiver uses the control to record a particular patient's weight. The controller is further adapted to thereafter automatically perform the following: (i) when the caregiver has selected the first patient monitoring technique, compare a current patient weight to the initial patient weight and provide a notification to the caregiver if the current patient weight differs from the initial patient weight by more than an adaptive threshold; and (ii) when the caregiver has selected the second patient monitoring technique, compare a current patient weight to the initial patient weight and provide the notification to the caregiver if the current patient weight differs from the initial patient weight by more than a non-adaptive threshold.

The controller, in some aspects, is adapted to select a value for the adaptive threshold that is based on fluctuations in the patient's weight over a time period.

In some aspects, the controller is further adapted to determine a mean absolute deviation of the fluctuations and to select the value for the adaptive threshold based on the mean absolute deviation of the fluctuations.

In some aspects, the controller is further adapted to select a value for the adaptive threshold that is greater than the mean absolute deviation.

The controller, in some aspects, is further adapted to increase the time period.

The patient support apparatus, in some aspects, further includes a new patient control adapted to be activated by the caregiver when a new patient is assigned to the patient support apparatus. The controller is further adapted to erase the initial patient weight of the particular patient when the new patient control is activated.

The controller, in some aspects, is further adapted to store in the memory a new initial patient weight corresponding to a first time the caregiver uses the control to record a new patient's weight, and to thereafter perform the following: (i) when the caregiver has selected the first patient monitoring technique, compare a current weight of the new patient to the new initial patient weight and provide the notification to the caregiver if the current weight of the new patient differs from the new initial patient weight by more than the adaptive threshold; and (ii) when the caregiver has selected the second patient monitoring technique, compare a current weight of the new patient to the new initial patient weight and provide the notification to the caregiver if the current weight of the new patient differs from the new initial patient weight by more than the non-adaptive threshold.

The controller, in some aspects, is further adapted to display a patient weight gain/loss indicator on the display that provides an indication to the caregiver of a difference between the current patient weight and a last saved patient weight. The last saved patient weight corresponds to a weight of the patient recorded when the caregiver most recently activated the control.

In some aspects, the patient support apparatus further includes a network transceiver adapted to communicate with a server hosted on a healthcare facility computer network. The controller may be further adapted to send a notification message to the server if either if following occurs: (i) the caregiver has selected the first patient monitoring technique and the current patient weight differs from the initial patient weight by more than the adaptive threshold; or (ii) the caregiver has selected the second patient monitoring technique and the current patient weight differs from the initial patient weight by more than the non-adaptive threshold.

In some aspects, the controller is further adapted to display a threshold selection screen on the display. The threshold selection screen is adapted to allow the caregiver to adjust the non-adaptive threshold.

The threshold selection screen, in some aspects, includes a slider icon displayed thereon adapted to be repositioned by the caregiver to enable the caregiver to select the non-adaptive threshold.

In some aspects, the slider icon is adapted to slide within a range of values for the non-adaptive threshold that includes values between at least one and two kilograms.

The slider icon, in some aspects, may specify a percentage of the patient's weight.

In some aspects, the controller is adapted to display a cumulative weight change of the patient wherein the cumulative weight change corresponds to a change in the patient's weight between a time when the patient's weight is first measured on the patient support apparatus and the present time.

In some aspects, the controller displays both the cumulative weight change of the patient and a most recent weight change, wherein the most recent weight change corresponds to a change in the patient's weight between the last time the patient's weight was recorded and the present time.

According to another aspect of the present disclosure, a patient support apparatus is provided that includes a support surface, a plurality of force sensors, a memory, a high precision control, a display, and a controller. The support surface is adapted to support a patient thereon. The plurality of force sensors are adapted to detect downward forces exerted on the support surface. The controller is adapted to determine a current weight of the patient supported on the support surface based on outputs from the plurality of force sensors. The controller is further adapted to display, by default, the current weight of the patient on the display with a low level of precision, and to temporarily display the current weight of the patient on the display with a higher level of precision in response to a caregiver activating the high precision control.

According to other aspects of the present disclosure, the controller is further adapted to display a patient weight gain/loss indicator on the display. The patient weight gain/loss indicator provides an indication to the caregiver of a difference between the current patient weight and a last saved patient weight. The controller is further adapted, by default, to display the patient weight gain/loss indicator with the low level of precision and to temporarily display the patient weight gain/loss indicator with the higher level of precision in response to the caregiver activating the high precision control.

In some aspects, the controller is further adapted to display an object weight log indicator on the display that indicates a total weight of objects that have been added to the patient support, and the controller is further adapted, by default, to display the object weight log indicator with the low level of precision and to temporarily display the object weight log indicator with the higher level of precision in response to the caregiver activating the high precision control.

The controller, in some aspects, is adapted to display the current weight of the patient with the higher level of precision for a fixed amount of time after the caregiver activates the high precision control.

In some aspects, the high precision control is a touchscreen control displayed on the display, and the controller is adapted to display an icon at the location of the touchscreen control and to temporarily change an appearance of the icon after the caregiver activates the high precision control.

The controller, in some aspects, is adapted to change the appearance of the icon by displaying a varying portion of a circle at the icon.

The controller, in some aspects, is adapted to vary the portion of the circle until the fixed amount of time expires.

The controller may further be adapted to decrease the portion of the circle during the fixed amount of time such that the portion of the circle disappears when the fixed amount of time expires.

Before the various embodiments disclosed herein are explained in detail, it is to be understood that the claims are not to be limited to the details of operation or to the details of construction and the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to one embodiment of the disclosure;

FIG. 2 is a perspective view of a litter frame of the patient support apparatus;

FIG. 3 is a perspective view of a base of the patient support apparatus;

FIG. 4 is a plan view of a control panel that may be included with the patient support apparatus;

FIG. 5 is a block diagram of a control system of the patient support apparatus, as well as several external devices with which the patient support apparatus may be configured to communicate;

FIG. 6 is an illustrative scale screen displayable on the control panel of FIG. 4 illustrating a situation where a cumulative patient change is less than a threshold;

FIG. 7 is an illustrative scale screen displayable on the control panel of FIG. 4 illustrating a situation where the cumulative patient change is greater than the threshold;

FIG. 8 is an illustrative scale screen displayable on the control panel of FIG. 4 that includes a display of the patient's recent weight change and the patient's cumulative weight change;

FIG. 9 is an illustrative threshold selection screen displayable on the control panel of FIG. 4 that includes a selection for adaptive and non-adaptive thresholds, as well as a slider control for selecting a value of a non-adaptive threshold;

FIG. 10 is an illustrative scale zeroing and new patient screen that allows a caregiver to inform the patient support apparatus that a new patient has been assigned to it;

FIG. 11 is another illustrative scale screen displayable on the control panel of FIG. 4 in some embodiments of the patient support apparatus that permits a user to temporarily access a higher resolution weight reading; and

FIG. 12 is the illustrative scale screen of FIG. 11 showing temporary higher resolution weight readings.

### DETAILED DESCRIPTION OF THE EMBODIMENTS

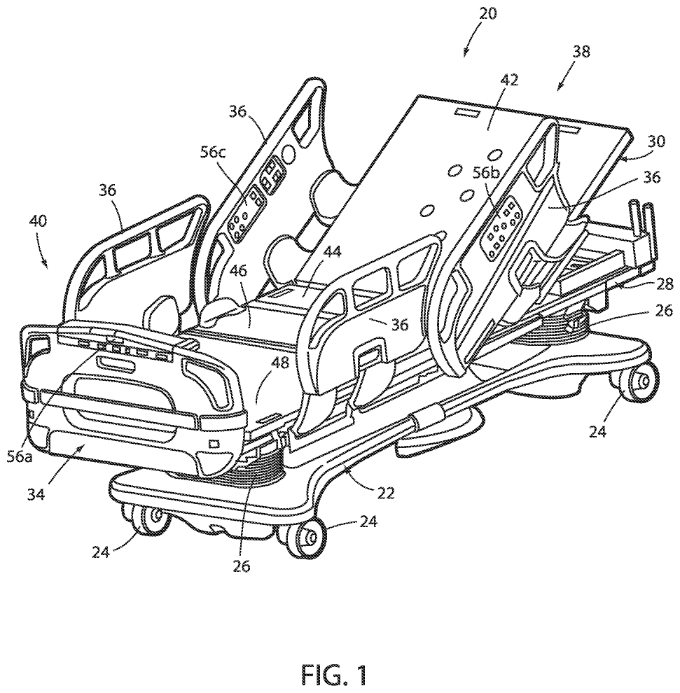

An illustrative patient support apparatus 20 that may incorporate one or more aspects of the present disclosure is shown in FIG. 1. Although the particular form of patient support apparatus 20 illustrated in FIG. 1 is a bed adapted for use in a hospital or other medical setting, it will be understood that patient support apparatus 20 could, in different embodiments, be a cot, a stretcher, a gurney, a recliner, a residential bed, or any other structure capable of supporting a patient, whether stationary or mobile and/or whether medical or residential.

In general, patient support apparatus 20 includes a base 22 having a plurality of wheels 24, a pair of lifts 26 supported on the base, a litter frame 28 supported on the lifts 26, and a support deck 30 supported on the litter frame 28. Patient support apparatus 20 further includes a footboard 34, and a plurality of siderails 36. Siderails 36 are all shown in a raised position in FIG. 1 but are each individually movable to a lower position in which ingress into, and egress out of, patient support apparatus 20 is not obstructed by the lowered siderails 36. In some embodiments, siderails 36 may be moved to one or more intermediate positions as well.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated embodiment, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, footboard 34, and siderails 36. Support deck 30 provides a support surface for a mattress (not shown), or other soft cushion, so that a person may lie and/or sit thereon. Support deck 30 is made of a plurality of sections, some of which are pivotable about generally horizontal pivot axes. In the embodiment shown in FIG. 1, support deck 30 includes at least a head section 42, a seat section 44, a thigh section 46, and a foot section 48, all of which generally form flat surfaces for supporting the mattress. Head section 42, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some embodiments, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width. Notionally, the first standard width may be considered a 36 inch width, the second intermediate width may be considered a 42 inch width and the third more expanded width may be considered a 48 inch width, although these numerical widths may be varied to comprise different width values.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another embodiment, the mechanical construction of patient support apparatus 20 may be the same as, or nearly the same as, the mechanical construction of the Model 3002 S3 bed manufactured and sold by Stryker Corporation of Kalamazoo, Michigan. This mechanical construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. It will be understood by those skilled in the art that patient support apparatus 20 can be designed with still other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on still other forms different from what is disclosed in the aforementioned references.

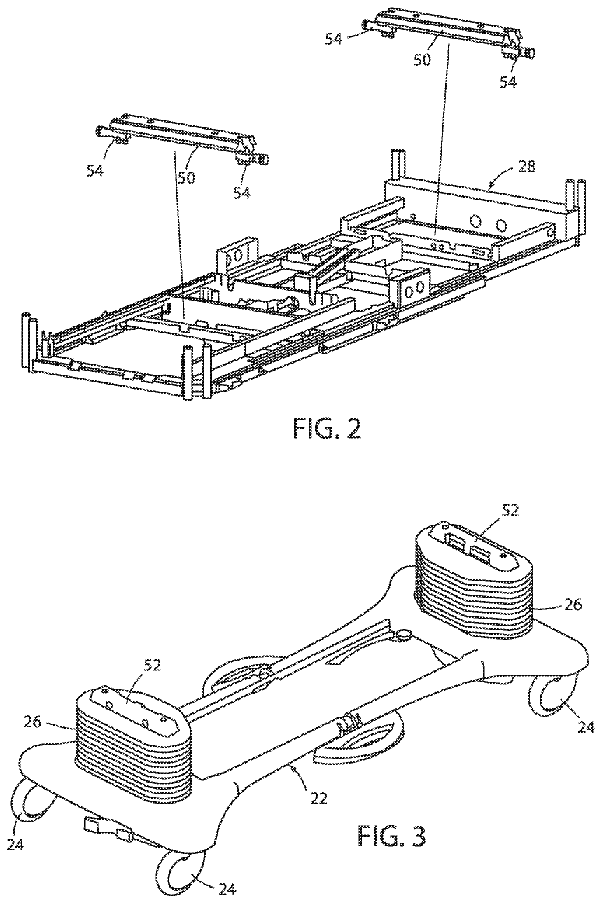

FIG. 2 illustrates in greater detail litter frame 28 separated from lifts 26 and base 22. Litter frame 28 is also shown in FIG. 2 with support deck 30 removed. Litter frame 28 is supported by two lift header assemblies 50. A first one of the lift header assemblies 50 is coupled to a top 52 (FIG. 3) of a first one of the lifts 26, and a second one of the lift header assemblies 50 is coupled to the top 52 of the second one of the lifts 26. Each lift header assembly 50 includes a pair of force sensors 54, which may be load cells, or other types of force sensors. The illustrated embodiment of patient support apparatus 20 therefore includes a total of four force sensors 54, although it will be understood by those skilled in the art that different numbers of force sensors may be used in accordance with the principles of the present disclosure. Force sensors 54 are configured to support litter frame 28. More specifically, force sensors 54 are configured such that they provide complete and exclusive mechanical support for litter frame 28 and all of the components that are supported on litter frame 28 (e.g. support deck 30, footboard 34, the headboard, siderails 36, etc.). Because of this construction, force sensors 54 are adapted to detect the weight of not only those components of patient support apparatus 20 that are supported by litter frame 28 (including litter frame 28 itself), but also any objects or persons who are wholly or partially being supported by support deck 30. The outputs of force sensors 54 are fed into a scale system described in greater detail below.

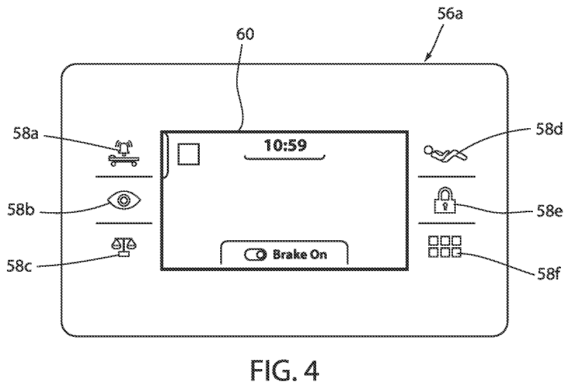

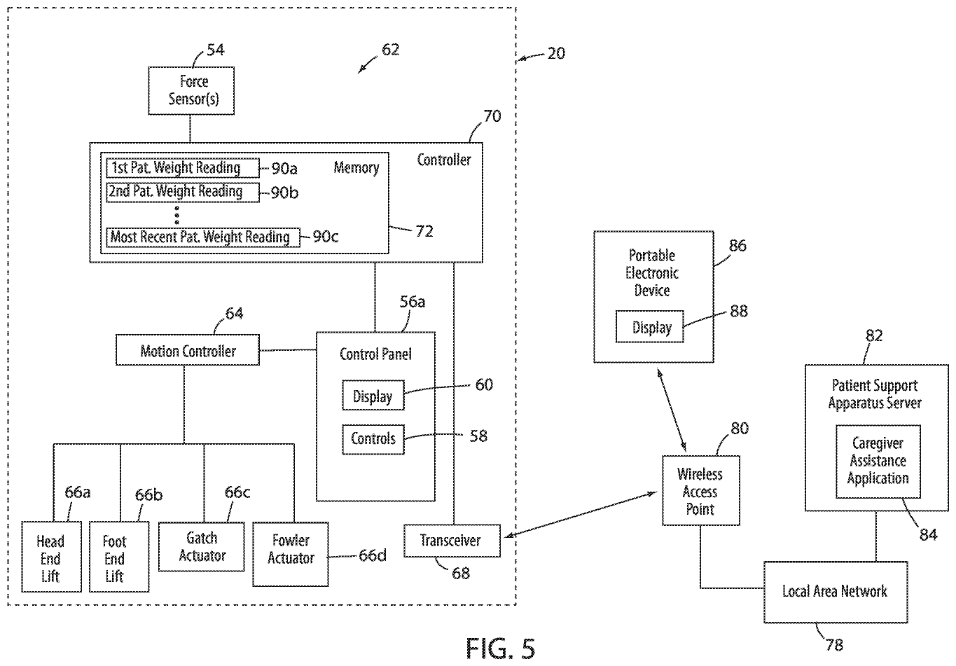

Patient support apparatus 20 further includes a plurality of control panels 56 (FIG. 1) that enable a user of patient support apparatus 20, such as a patient and/or an associated caregiver, to control one or more aspects of patient support apparatus 20. In the embodiment shown in FIG. 1, patient support apparatus 20 includes a footboard control panel 56a, a pair of outer siderail control panels 56b (only one of which is visible), and a pair of inner siderail control panels 56c (only one of which is visible). Footboard control panel 56a concealed by a lid in FIG. 1, but is otherwise visible and accessible to a caregiver when the lid is lifted. In some embodiments, the lid is eliminated from patient support apparatus 20 and the control panel 56a is always visible. Footboard control panel 56a and outer siderail control panels 56b are intended to be used by caregivers, or other authorized personnel, while inner siderail control panels 56c are intended to be used by the patient associated with patient support apparatus 20. Each of the control panels 56 includes a plurality of controls 58 (see, e.g. FIGS. 4-5), although each control panel 56 does not necessarily include the same controls and/or functionality.

Among other functions, controls 58 of control panel 56a allow a user to control one or more of the following: change a height of support deck 30, raise or lower head section 42, activate and deactivate a brake for wheels 24, take measurements of the patient's weight, arm and disarm one or more patient monitoring functions, change various settings on patient support apparatus 20, view the current location of the patient support apparatus 20 as determined by a location detection system, and perform still other actions. One or both of the inner siderail control panels 56c also include at least one control 58 that enables a patient to call a remotely located nurse (or other caregiver).

Control panel 56a includes a display 60 (FIG. 4) configured to display a plurality of different screens thereon. Surrounding display 60 are a plurality of navigation controls 58a-f that, when activated, cause the display 60 to display different screens on display 60. For example, when a user presses navigation control 58a, control panel 56a displays an exit detection control screen on display 60 that includes one or more icons that, when touched, control an onboard exit detection system. The exit detection system is adapted to issue an alert when a patient exits from patient support apparatus 20. Such an exit detection system may include any of the same features and/or functions as, and/or may be constructed in any of the same manners as, the exit detection systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and/or the exit detection system disclosed in commonly assigned U.S. Pat. No. 5,276,432 issued to Travis and entitled PATIENT EXIT DETECTION MECHANISM FOR HOSPITAL BED, the complete disclosures of both of which are incorporated herein by reference.

When a user presses navigation control 58b (FIG. 4), control panel 56a displays a patient support apparatus monitoring control screen that includes a plurality of control icons that, when touched, control an onboard monitoring system that monitors one or more components, features, and/or other aspects of patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference. Other types of monitoring systems may be included within patient support apparatus 20 for monitoring parameters of the patient support apparatus 20.

When a user presses navigation control 58c, control panel 56a displays a scale control screen that includes a plurality of control icons that, when touched, control the scale system of patient support apparatus 20. In addition to various features of the scale system that are discussed in greater detail below, the scale system of patient support apparatus 20 may include any of the same features, components, and/or and functions as the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference. The scale system may utilize the same force sensors that are utilized by the exit detection system, in some embodiments, or it may utilize one or more different sensors. Further details regarding the scale system are described in greater detail below.

When a user presses navigation control 58d, control panel 56 displays a motion control screen that includes a plurality of control icons that, when touched, control the movement of various components of patient support apparatus 20, such as, but not limited to, the height of litter frame 28 and the pivoting of head section 42. In some embodiments, the motion control screen displayed on display 60 in response to pressing control 58d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of motion control screens may be included on patient support apparatus 20.

When a user presses navigation control 58e (FIG. 4), control panel 56a displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosure of which is incorporated herein by reference. Other types of motion lockouts may be included within patient support apparatus 20.

When a user presses on navigation control 58f, control panel 56a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, location information, medical device association information, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference. Other types of menus and/or settings may be included within patient support apparatus 20. In at least one embodiment, utilization of navigation control 58f allows a user to navigate to a screen that enables a user to configure the communication settings between patient support apparatus 20 and one or more wall units. Examples of the type of communication settings that may be configured in this manner are disclosed in, and illustrated in FIGS. 9-15 of, commonly assigned U.S. patent application Ser. No. 63/26,937 filed May 19, 2020, by inventors Alexander Bodurka et al. and entitled PATIENT SUPPORT APPARATUSES WITH HEADWALL COMMUNICATION, the complete disclosure of which is incorporated herein by reference.

For all of the navigation controls 58a-f (FIG. 4), screens other than the ones specifically mentioned above may be displayed on display 60 in other embodiments of patient support apparatus 20 in response to a user pressing these controls. Thus, it will be understood that the specific screens mentioned above are merely representative of the types of screens that are displayable on display 60 in response to a user pressing on one or more of navigation controls 58a-f. It will also be understood that, although navigation controls 58a-f have all been illustrated in the accompanying drawings as dedicated controls that are positioned adjacent display 60, any one or more of these controls 58a-f could alternatively be touchscreen controls that are displayed at one or more locations on display 60. Still further, although controls 58a-f have been shown herein as buttons, it will be understood that any of controls 58a-f could also, or alternatively, be switches, dials, or other types of non-button controls.

As shown in FIG. 5, patient support apparatus 20 includes a control system 62 that controls various aspects of patient support apparatus 20. Control system 62 includes control panels 56 (only one of which—56a—is shown in FIG. 5), force sensors 54, a motion controller 64, a plurality of motorized actuators 66, a network transceiver 68, and a main controller 70. Main controller 70 includes a memory 72. Main controller 70 and force sensors 54 together act as both a scale system and an exit detection system. When acting as an exit detection system, main controller 70 and force sensors 54 are adapted to determine when an occupant leaves patient support apparatus, or moves in a way indicative of an imminent departure from the patient support apparatus. In such situations, main controller 70 is configured to issue an alert and/or notification to appropriate personnel so that proper steps can be taken in response to the occupant's departure, or imminent departure, in a timely fashion.

Force sensors 54 are adapted to detect downward forces exerted by an occupant of support deck 30. Thus, when an occupant is positioned on support deck 30 and remains substantially still (i.e. not moving in a manner involving accelerations that cause forces to be exerted against support deck 30), force sensors 54 will detect the weight of the occupant (as well as the weight of any components of patient support apparatus 20 that are supported—directly or indirectly—by force sensors 54). In at least one embodiment, force sensors 54 are load cells. However, it will be understood by those skilled in the art, that force sensors 54 may be implemented as other types of sensors, such as, but not limited to, linear variable displacement transducers and/or any one or more capacitive, inductive, and/or resistive transducers that are configured to produce a changing output in response to changes in the force exerted against them.

Main controller 70 and motion controller 64 are constructed of any electrical component, or group of electrical components, that are capable of carrying out the functions described herein. In many embodiments, controllers 64 and 70 are conventional microcontrollers, although not all such embodiments need include a microcontroller. In general, controllers 64 and 70 include any one or more microprocessors, microcontrollers, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. Indeed, in some embodiments, main controller 70 and motion controller 64 are combined with each other and/or with other circuitry or controllers that are present on patient support apparatus 20. The instructions followed by controllers 64 and 70 in carrying out the functions described herein, as well as the data necessary for carrying out these functions, are stored in one or more memories that are accessible to them (e.g. memory 72 for main controller 70).

Although patient support apparatus 20 includes a total of four force sensors 54, it will be understood by those skilled in the art that different numbers of force sensors 54 may be used in accordance with the principles of the present disclosure. Force sensors 54, in at least one embodiment, are configured to support litter frame 28. When so configured, force sensors 54 are constructed to provide complete and exclusive mechanical support for litter frame 28 and all of the components that are supported on litter frame 28 (e.g. deck 30, footboard 34, and, in some embodiments, siderails 36). Because of this construction, force sensors 54 are adapted to detect the weight of not only those components of patient support apparatus 20 that are supported by the litter frame 28 (including litter frame 28 itself), but also any objects or persons who are positioned either wholly or partially on support deck 30. By knowing the weight of the components of the patient support apparatus 20 that are supported on litter frame 28, controller 70 is able to determine a tare weight that, when subtracted from a total weight sensed after a patient is supported on support deck 30, yields a patient weight.

In some embodiments, the physical location of the force sensors 54 on patient support apparatus 20 may be modified to be located on the base frame, such as shown in commonly assigned U.S. patent application Ser. No. 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference. In other embodiments, the physical location of the force sensors 54 on patient support apparatus 20 may be the same as the position of the load cells disclosed in commonly assigned U.S. patent application Ser. No. 15/266,575 filed Sep. 15, 2016, by inventors Anuj Sidhu et al. and entitled PERSON SUPPORT APPARATUSES WITH EXIT DETECTION SYSTEMS, the complete disclosure of which is also incorporated herein by reference. In still other embodiments, the physical location of the force sensors 54 may be the same as the position of the load cells disclosed in U.S. Pat. No. 7,962,981 issued to Lemire et al. and entitled HOSPITAL BED, the complete disclosure of which is also incorporated herein by reference. In still other embodiments, force sensors 54 may be positioned on patient support apparatus 20 at still other locations.

Motion controller 64 (FIG. 5) is adapted to control the movement of a plurality of components of patient support apparatus 20. These components includes, but are not limited to, a head end lift actuator 66a, a foot end lift actuator 66b, a gatch actuator 66c, and a Fowler actuator 66d. Each of these actuators 66a-d may comprise a linear actuator with a motor built therein. In some embodiments, the linear actuator may be of the type disclosed in commonly assigned U.S. patent application Ser. No. 15/449,277 filed Mar. 3, 2017, by inventors Anish Paul et al. and entitled PATIENT SUPPORT APPARATUS WITH ACTUATOR FEEDBACK, the complete disclosure of which is incorporated herein by reference. In other embodiments, other types of powered actuators may be used, such as, but not limited to, hydraulic and/or pneumatic actuators.

In some embodiments, motion controller 64 operates in the same or similar manners to the main microcontroller 58 and its associated circuitry disclosed in commonly assigned U.S. Pat. No. 10,420,687 issued Sep. 24, 2019, to inventors Aaron Furman et al. and entitled BATTERY MANAGEMENT FOR PATIENT SUPPORT APPARATUSES, the complete disclosure of which is incorporated herein by reference. In such embodiments, motion controller 64 controls the sending of pulse width modulated (PWM) signals to the motors contained within actuators 66a-d, thereby controlling both the speed and the direction of movement of these actuators. Motion controller 64 may take on other forms as well.

Motion controller 64 is in communication with control panel 56 and receives signals from control panel 56 indicating when a user wishes to move one or more components of patient support apparatus 20. That is, control panel 56 includes one or more controls 58 that are adapted, when activated, to instruct motion controller 64 to carry out the desired movement of the various movable components of patient support apparatus 20, as well as one or more controls for stopping such motion. Such movement includes, but is not limited to, raising and lowering the height of litter frame 28, pivoting the Fowler section 42 up and down about a generally horizontal axis (extending laterally from one side of the patient support apparatus 20 to the other), and/or lifting and lowering a knee gatch on patient support apparatus 20.

Head end lift actuator 66a is configured to change the height of the head end 38 of litter frame 28. Foot end lift actuator 66b is configured to change the height of the foot end 40 of litter frame 28. When both of these actuators 66a and 66b are operated simultaneously and at the same speed, the height of litter frame 28 is raised or lowered without changing the general orientation of litter frame 28 with respect horizontal. When one or more of these actuators 66a and/or 66b are operated at different times and/or at different speeds, the orientation of litter frame 28 is changed with respect to horizontal. Lift actuators 66a and 66b are therefore able to tilt litter frame 28 to a variety of different orientations, including, but not limited to, a Trendelenburg orientation and a reverse-Trendelenburg orientation.

Gatch actuator 66c is adapted to raise and lower the joint that couples together the thigh section 46 and the foot section 48 of support deck 30, thereby raising and lowering the portion of the support deck 30 that is positioned close to the patient's knees. Fowler actuator 66d is adapted to raise and lower the head section (or Fowler section) 42 of the support deck 30.

Control panel 56 (FIG. 4) communicates with main controller 70 and is adapted to allow a user to control the various functions performed by main controller 70, including, but not limited to, the scale function and the exit detection function carried out by main controller 70 (and force sensors 54). The plurality of controls 58 of control panel 56 may be implemented as buttons, dials, switches, icons on a touchscreen, or other devices. Display 60 of control panel 56 may be a touchscreen that displays one or more controls and/or one or more control screens, some of which are discussed in greater detail below. Display 60 may comprise an LED display, OLED display, or another type of display.

Control system 62 may include additional components beyond those shown in FIG. 5. Alternatively, or additionally, one or more of the components shown in FIG. 5 may be omitted from patient support apparatus 20, or replaced by other components. In some embodiments, control system 62 is configured to automatically maintain a weight log of non-patient items added to, and removed from, the patient support apparatus, thereby making it easier for the caregiver to take accurate readings of the patient's weight. In some such embodiments, control system 62 includes any of the additional components and/or functionality of the scale systems disclosed in the following commonly assigned U.S. patent references: U.S. patent application Ser. No. 63/255,211 filed Oct. 13, 2021, by inventors Sujay Sukumaran et al. and entitled PATIENT SUPPORT APPPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. Pat. No. 11,033,233 issued to Michael Hayes et al. on Jun. 15, 2021, and entitled PATIENT SUPPORT APPARATUS WITH PATIENT INFORMATION SENSORS; and U.S. patent application Ser. No. 16/992,515 filed Aug. 13, 2020, by inventors Kurosh Nahavandi et al. and entitled PATIENT SUPPORT APPARATUS WITH EQUIPMENT WEIGHT LOG, the complete disclosures of all of which are incorporated herein by reference.

Network transceiver 68 (FIG. 5) is adapted to communicate with a hospital's local area network 78. In some embodiments, network transceiver 68 is a conventional WiFi transceiver (i.e. IEEE 802.11 . . . ) adapted to wirelessly communicate with one or more wireless access points 80 of a hospital's local area network 78. In other embodiments, network transceiver 68 may be a wireless transceiver that uses conventional 5G technology to communicate with network 78, one or more servers hosted thereon, and/or other devices. In some embodiments, network transceiver 68 may include any of the structures and/or functionality of the communication modules 56 disclosed in commonly assigned U.S. Pat. No. 10,500,401 issued to Michael Hayes and entitled NETWORK COMMUNICATION FOR PATIENT SUPPORT APPARATUSES, the complete disclosure of which is incorporated herein by reference. Still other types of wireless network transceivers may be utilized.

In other embodiments, network transceiver 68 may be a conventional Ethernet transceiver electrically coupled to a conventional Ethernet port (i.e. RJ-45 jack, or the like) built into patient support apparatus 20 that allows a conventional Ethernet cable to be coupled to the patient support apparatus 20. In these embodiments, patient support apparatuses 20 may be coupled to the hospital's local area network 78 by a wired connection. In still other embodiments, patient support apparatus 20 may have both wired and wireless transceivers 68. Still further, in some embodiments, transceiver 68 may take on yet a different form (e.g. a wireless ZigBee transceiver, a Bluetooth transceiver, etc.).

Patient support apparatus 20 uses transceiver 68, in some embodiments, to communicate with a patient support apparatus server 82. Patient support apparatus server 82 may be adapted to receive status information from patient support apparatuses 20 and distribute that information to one or more other servers and/or other devices coupled to local area network 78. In at least one embodiment, patient support apparatus server 82 includes a caregiver assistance software application 84 that is adapted to communicate information between both patient support apparatuses 20 and one or more portable electronic devices 86. The portable electronic devices 86 includes, but are not limited to, smart phones, tablets, laptops, Computers on Wheels (COWs), and the like. Each portable electronic device 86 includes a display 88 on which various screens may be displayed, including, in some embodiments, portions of one or more of the screens discussed below. In some embodiments, caregiver assistance application 84 allows authorized users to remotely configure and remotely control various aspects of the patient support apparatuses 20 using their portable electronic device 86. Still further, caregiver assistance application 84 may be adapted to display information about the scale systems of the patient support apparatuses 20, including any of the information discussed in greater detail below regarding the scale system.

In any of the embodiments disclosed herein, caregiver assistance application 84 (FIG. 5) may be configured to include any of the same features or functions as—and/or to operate in any of the same manners as—the caregiver assistance software applications described in the following commonly assigned patent applications: U.S. patent application Ser. No. 62/826,097, filed Mar. 29, 2019 by inventors Thomas Durlach et al. and entitled PATIENT CARE SYSTEM; U.S. patent application Ser. No. 16/832,760 filed Mar. 27, 2020, by inventors Thomas Durlach et al. and entitled PATIENT CARE SYSTEM; and/or PCT patent application serial number PCT/US2020/039587 filed Jun. 25, 2020, by inventors Thomas Durlach et al. and entitled CAREGIVER ASSISTANCE SYSTEM, the complete disclosures of which are all incorporated herein by reference. Thus, server 82 may be configured to share with one or more electronic devices 86 any of the information shared with the electronic devices disclosed in these aforementioned patent applications. For example, server 82 may be configured to not only share the location of patient support apparatuses 20 (and any devices that may be associated with them) with electronic devices 86, but it may also forward any of the data generated by patient support apparatuses 20 to the electronic devices 86, thereby letting the caregivers associated with these patient support apparatuses 20 know if, for example, the patient has exited patient support apparatus 20, what the patient's current weight is, whether the patient's cumulative weight change has exceeded a threshold or not, etc. Alternatively, or additionally, patient support apparatus server 82 may forward other patient support apparatus status data (e.g. current siderail position, bed exit status, brake status, height status, scale data, etc.) and/or caregiver rounding information (e.g. when the last rounding was performed for a particular patient, when the next rounds are due, etc.), and/or object data from any objects supported on patient support apparatus 20 to one or more electronic devices 86, thereby providing the caregivers associated with the devices 86 a consolidated portal (e.g. a single software application) for sharing this various information.

It will be understood that, although caregiver assistance application 84 is shown in FIG. 5 to be executed solely by server 82, this may be modified so that caregiver assistance application 84 is executed, either partially or wholly, on one or more of the mobile devices 86 as a mobile app that is downloaded to these devices 86. Similarly, caregiver assistance application 84 may be implemented as an app that executes on server 82 in conjunction with another specialized app that is downloaded to each of the mobile electronic devices 86. Still other variations of caregiver assistance application 84 are possible.

In some embodiments, patient support apparatus server 82 is also configured to determine the location of each patient support apparatus 20, or receive the location of each patient support apparatus 20 from the patient support apparatuses 20. In some embodiments, patient support apparatus server 82 determines the room number and/or bay area of each patient support apparatus 20 that is positioned within a room, as well as the location of patient support apparatuses 20 that are positioned outside of a room, such as, those that may be positioned in a hallway, a maintenance area, or some other area. In general, patient support apparatus server 82 may be configured to determine the position of any patient support apparatus 20 by communicating with one or more nearby wall units (not shown). Further details regarding several manners in which patient support apparatus 20 may be constructed in order to carry out such location communication, as well as the construction and/or operation of such wall units, are disclosed in the following commonly assigned U.S. patent applications: Ser. No. 63/245,245 filed Sep. 17, 2021, by inventors Kirby Niehouser et al., and entitled SYSTEM FOR LOCATING PATIENT SUPPORT APPARATUSES; Ser. No. 63/245,289 filed Sep. 17, 2021, by inventors Madhu Sandeep Thota et al. and entitled PATIENT SUPPORT APPARATUS COMMUNICATION AND LOCATION SYSTEM; Ser. No. 63/026,937 filed May 19, 2020, by inventors Alexander Bodurka et al. and entitled PATIENT SUPPORT APPARATUSES WITH HEADWALL COMMUNICATION; and Ser. No. 63/193,778 filed May 27, 2021, by inventors Krishna Bhimavarapu et al. and entitled PATIENT SUPPORT APPARATUS AND HEADWALL UNIT SYNCHING; the complete disclosures of all of which have already been incorporated herein by reference.

It will be understood that the architecture and content of local area network 78 will vary from healthcare facility to healthcare facility, and that FIG. 5 merely shows a generic example of the type of network a healthcare facility may be employ. Typically, one or more additional servers will be hosted on network 78 and one or more of them may be adapted to communicate with patient support apparatus server 82. For example, an electronic health record server will typically be present in any healthcare facility, and in some embodiments discussed herein, it will be in communication with patient support apparatus server 82 in order to receive patient data that is to be recorded in a patient's health record (e.g. weight readings taken from the scales built into patient support apparatuses 20; therapies provided to patients using a powered mattress onboard patient support apparatuses 20, etc.).

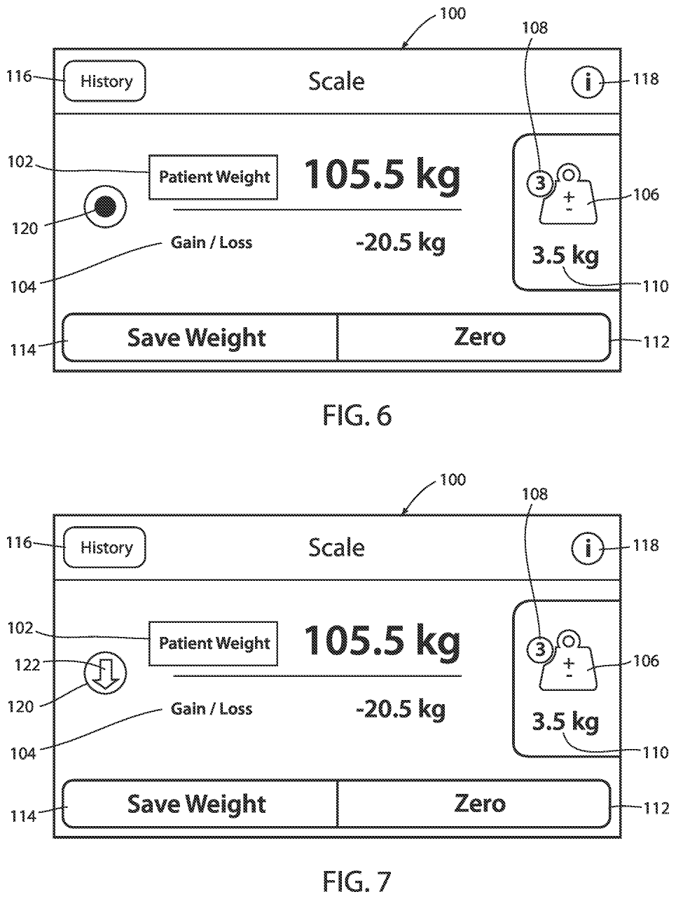

FIG. 6 illustrates a scale screen 100 that controller 70 may be configured to display on display 60 in response to the user pressing on scale control 58c. Scale screen 100 includes a patient weight indicator 102, a gain/loss indicator 104, a weight log icon 106, an object number 108, an object total weight indicator 110, a zero control 112, a save weight control 114, a scale history control 116, an information icon 118, and a cumulative patient weight change indicator 120. Patient weight indicator 102 indicates the currently measured patient weight, which is determined by subtracting the weight of any objects that have been placed on patient support apparatus 20 and entered by controller 70 into a weight log from the current total weight on force sensors 54 (after the force sensors have been zeroed). Gain/loss indicator 104 indicates the total patient weight loss or gain since the patient's weight was last saved by the user. As is discussed in more detail below, controller 70 is configured to store in memory 72 each of the patient weight readings that a caregiver saves for a particular patient. The gain/loss indicator 104 displays the difference between the current patient weight and the last saved patient weight reading in memory 72.

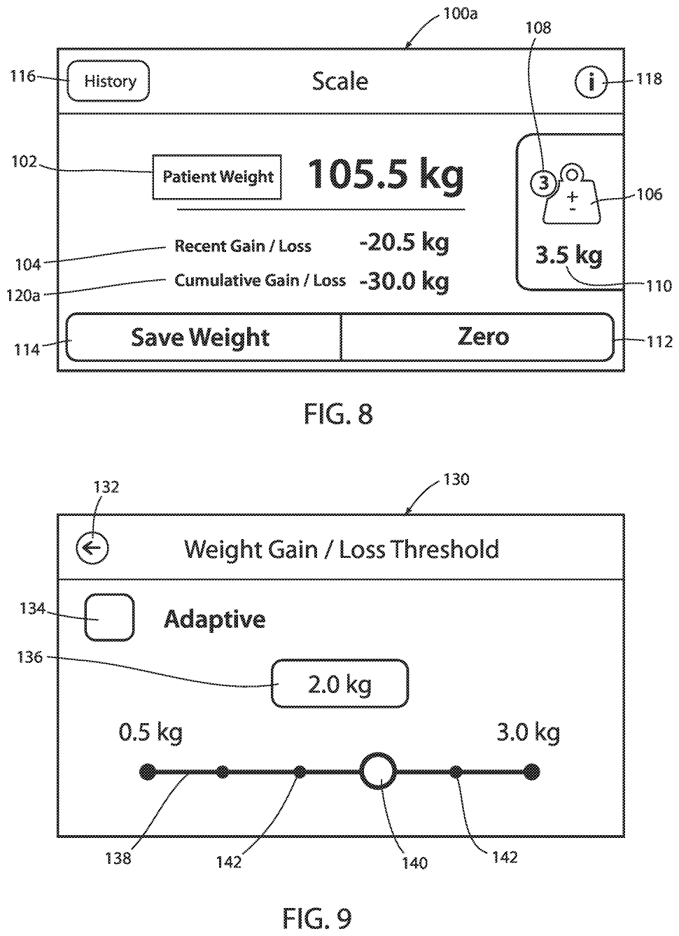

Log icon 106 is an icon that corresponds to a weight log that is maintained by controller 70 in some embodiments. That is, log icon 106 corresponds to a weight log that controller 70 may maintain of all of the objects that may be added to the litter frame 28 of patient support apparatus 20. If the user presses on weight log icon 106, controller 70 is configured to display a screen that provides more information about the weight log, such as a screen like those shown in FIGS. 8-11, 13, 15, and/or 16 of commonly assigned U.S. patent application Ser. No. 63/255,211 filed Oct. 13, 2021, by inventors Sujay Sukumaran et al. and entitled PATIENT SUPPORT APPARATUS WITH AUTOMATIC SCALE FUNCTIONALITY, the complete disclosure of which has already been incorporated herein by reference. Object number 108 indicates the number of objects that are currently in the weight log. Thus, in the example shown in FIG. 6, the weight log currently has three objects recorded therein that are positioned on litter frame 28. Object weight total indicator 110 indicates the total weight of the objects currently stored in the weight log. Thus, in the example of FIG. 6, the three objects stored in the weight log have a total cumulative weight of 3.5 kg, as indicated by indicator 110.

Screen 100 (FIG. 6) also includes a save weight control 114 and a zero control 112. If the user presses on the save weight control 114, controller 70 is configured to store the currently measured patient weight (as indicated by indicator 102) within memory 72. That stored patient weight is then used as a baseline for future patient weight readings when controller 70 computes the value to be shown by the gain/loss indicator 104. In other words, when the user presses the save weight control 114, controller 70 zeroes the gain/loss indicator 104. In the example of FIG. 6, after the user presses the save weight control 114, controller 70 would change indicator 104 to zero and thereafter only show non-zero values if the patient's weight deviated from 105.5 kg.

Every time a caregiver presses the save weight control 114, controller 70 is configured to store the currently measured patient weight in memory 72. This is illustrated more clearly in FIG. 5, which shows a plurality of patient weight readings 90 stored therein. In this particular example, memory 72 includes a first patient weight reading 90a, a second patient weight reading 90b, a most-recent patient weight reading 90c, and an unspecified number of patient weight readings between the second patient weight reading 90b and the most-recent patient weight reading 90c. First patient weight reading 90a corresponds to the initial patient weight reading that a caregiver saves for a particular patient after the patient is assigned to patient support apparatus 20. Thus, for example, after a patient is first admitted to a healthcare facility, or first assigned to a patient support apparatus 20, the very first time the caregiver uses the save weight control 114 for that particular patient, controller 70 stores the initial patient weight reading as the first patient weight reading 90a.

During the course of the patient's stay at the healthcare facility, the caregiver may periodically take additional patient weight readings 90 and save them via control 114. These additional saved patient weight readings correspond to the weight readings 90b, 90c shown in FIG. 5 (as well any that occur between readings 90b and 90c). The gain/loss indicator 104 displays a weight value that corresponds to the difference between the current patient weight reading (as shown by indicator 102) and the most-recently saved patient weight reading 90c. As will be discussed in greater detail below, the difference between the current patient weight reading and the initial patient weight reading 90a corresponds to the cumulative patient weight change.

If the user presses on zero control 112 (FIG. 6), controller 70 is configured to zero (i.e. tare) the scale system. That is, in response to control 112 being activated by a user, controller 70 takes a snapshot of the current total weight readings recorded by force sensors 54 and considers that weight value to be equal to the empty weight of patient support apparatus 20 (i.e. the weight on the scale system when no patient is present and no objects of interest are present). Typically, a caregiver uses control 112 to zero the scale system when the patient is absent, but a mattress, a pillow, bedding, and other standard items (if any) are present on the patient support apparatus 20. In this manner, the weight of the mattress, pillow, bedding, etc., as well as the structural weight of the support deck 30 and litter frame 28, are zeroed out. In some embodiments, after the user has zeroed the scale system using control 112, controller 70 is configured to erase any items that were stored in the weight log.

Scale screen 100 also includes a scale history control 116. When a user presses on control 116, controller 70 is configured to display a different screen that graphically shows a history of the patient's weight readings. The graph may have time on the X-axis and the patient's weight on the Y-axis. The patient weight history screen gives the caregiver a visual overview of changes in the patient's weight while they were assigned to that particular patient support apparatus 20. The weight readings that are displayed on the graph correspond to the weight readings 90 that are stored in memory 72. In some embodiments, scale history control 116 is displayed in a first color (and/or with a first configuration) when there is data contained within the scale history, and in a second color (and/or with a second configuration) when there is no data contained within the scale history. Thus, for example, if the caregiver has never saved a weight reading of the patient, controller 70 might display control 116 with a first color until the caregiver saves the first patient weight reading 90a, at which point controller 70 will switch to displaying control 116 in a second color so that the user knows that at least one previous patient weight reading has been taken.

Scale screen 100 (FIG. 6) may also include an information icon 118. Controller 70 is configured to display additional information about the scale system when icon 118 is pressed by a caregiver. In some embodiments, the additional information includes any one or more of the following items: the last time the scale system was zeroed using control 112, a current software version, a date when the scale system was last calibrated, an indication of whether the scale is automatically detecting the addition and removal of objects or not, and/or information regarding the scale's compliance with, and/or class within, the Non-Automatic Weighing Instruments (NAWI) scale standards required by the European Union and/or their member states.

Cumulative patient weight change indicator 120 (FIG. 6) provides a notification to the caregiver when the patient's weight, since the time the patient was first admitted to the healthcare facility and/or assigned to that particular patient support apparatus 20, has changed by more than a threshold. Cumulative patient weight change indicator 120 is therefore adapted to provide a notification to the caregiver when large weight changes occur that might not otherwise be apparent to the caregiver, and that may be of clinical interest to the caregiver. Such large cumulative weight changes may not be evident to the caregiver because gain/loss indicator 104 only displays a change in weight between the patient's current weight and the last saved (i.e. most recent) patient weight reading 90c. As a result, there are situations where a patient's weight might change dramatically over the course of a few days, yet the gain/loss indicator 104 may never provide an indication of a weight change more than half a kilogram. For example, if caregivers are saving patient weight readings 90 twice a day, and the patient's weight for each reading drops by no more than half a kilogram, the patient may end up losing a significant amount of weight over the course of a few days or a week (e.g. the patient may lose up to seven kilograms within a week). Such a seven kilogram weight loss, however, will never be indicated by patient weight indicator 102. Instead, indicator 102 will be reset to zero after each patient weight reading is saved. As a result, caregivers will have to consult a patient's medical records to see a patient's cumulative weight loss since being admitted to the healthcare facility. Accordingly, in order to avoid requiring caregivers to consult the patient's medical records, patient support apparatus 20 is configured with cumulative patient weight change indicator 120.

Cumulative patient weight change indicator 120 is configured to change to a different state, such as a different color, size, and/or shape, when controller 70 detects that the patient's current weight has changed by more than a threshold value from the patient's initial weight reading 90a. FIG. 7 illustrates one example of this change. FIG. 7 illustrates scale control screen 100 at a time when cumulative patient weight change indicator 120 has changed to a different color and includes an arrow indicator 122. Arrow indicator 122 either points in an up direction or a down direction, depending upon whether the patient's total cumulative weight has increased by more than the threshold, or decreased by more than the threshold. Thus, in the example of FIG. 7, the patient's total cumulative weight has decreased by more than the threshold. Accordingly, controller 70 has changed the cumulative patient weight change indicator 120 in FIG. 7 to include a downward pointing arrow 122.

It will be understood that the manner in which controller 70 informs a caregiver that the patient's cumulative weight change has exceeded the threshold may vary from embodiment to embodiment. For example, in some embodiments, controller 70 is configured to not display any cumulative patient weight change indicator 120 when the patient's cumulative weight change is less than the threshold, and to only display the cumulative patient weight change indicator 120 (with or without arrow 122) when the patient's cumulative weight change is greater than the threshold.

Controller 70 may also inform the caregiver of cumulative weight changes exceeding the threshold in the manner illustrated in FIG. 8. FIG. 8 illustrates a modified scale screen 100a in which the cumulative patient weight change indicator 120 has changed to a numeric indicator 120a. Numeric indicator 120a provides a numeric value that is equal to the patient's cumulative weight change. In the example shown in FIG. 8, numeric indicator 120a shows that the patient has lost a total of thirty kilograms since the patient's initial weight reading 90a. In some embodiments, controller 70 is configured to change the color of cumulative patient weight indicator 120a to a different color when the patient's cumulative weight change exceeds the threshold. In other embodiments, controller 70 may be configured to add an icon, or another indicator (such as, but not limited to arrow 122) to screen 100a when the patient's cumulative weight change exceeds the threshold. In still other embodiments, controller 70 may be configured to simply provide the numeric value of the patient's cumulative weight change without comparing it to a threshold, and instead leave it to the caregiver to see this numeric value and act appropriately.

FIG. 9 illustrates an example of a threshold selection screen 130 that is displayed by controller 70 on display 60 in some embodiments of patient support apparatus 20. Controller 70 may be configured to display threshold selection screen 130 in response to the user pressing on cumulative patient weight change indicator 120, and/or in response to the user pressing on other controls 58 of patient support apparatus 20. Regardless of how a user navigates to screen 130, screen 130 includes a back icon 132, an adaptive threshold selector 134, a threshold value indicator 136, a slider bar 138, and a current threshold indicator 140. In response to a caregiver pressing on the back icon 132, controller 70 is configured to display whatever screen it was previously displaying on display 60. Adaptive threshold selector 134 is adapted to allow a caregiver to choose between an adaptive threshold and a non-adaptive threshold. When the caregiver wishes to use an adaptive threshold, he or she presses on selector 134 and controller 70 is configured to put a check mark, or other icon, in selector 134 indicating that he or she has selected the adaptive threshold. If the caregiver thereafter presses the selector 134 again, controller 70 removes the check mark, or other icon, and switches back to using a non-adaptive threshold. Selector 134 therefore acts as a toggle switch, in at least one embodiment, toggling between an adaptive threshold and a non-adaptive threshold.

If the caregiver selects the non-adaptive threshold via selector 134, screen 130 allows the user to select a numeric value for the non-adaptive threshold. Numeric value indicator 136 displays the currently selected numeric value for the threshold. In the example of FIG. 9, the current value is two kilograms. Slider bar 138 is adapted to allow the user to change the value of the threshold by sliding a current threshold indicator 140 along the length of slider bar 138. Thus, if the user slides indicator 140 all the way to the left end of slider bar 138, controller 70 will display a value of 0.5 kilograms at numeric value indicator 136 and use that value in its comparisons of the patient's cumulative weight change to his or her current weight. If the user slides indicator 140 all the way to the right end of slider bar 138, controller 70 will display a value of 3.0 kilograms at numeric value indicator 136 and use that value in its comparisons of the patient's cumulative weight change to his or her current weight.

Slider bar 138 (FIG. 9) includes a plurality of gradations 142 at different values. Gradations 142 provide visual feedback to the user of where current threshold indicator 140 is currently located along the slider bar 138. In the example shown in FIG. 9, a gradation indicator 142 is provided for every half kilogram along slider bar 138. Other spacing between gradations 142 may, or course, be used. Still further, other ranges for slider bar 138 may be used that include values higher than three kilograms and/or that include values lower than 0.5 kilograms along slider bar 138.

In some alternative embodiments, controller 70 is configured to modify slider bar 138 so that, instead of displaying absolute values of weight, it displays percentages. In such a case, the user is able to slide current threshold indicator 140 to a desired percentage, rather than to a desired weight. When implemented in this fashion, controller 70 uses a percentage of the patient's initial weight reading 90a as the threshold for its comparisons. For example, if the user selects a value of two percent along slider bar 138, and the patient's initial weight reading 90a is 100 kilograms, controller 70 will provide a notification to the user (e.g. via indicator 120 and arrow 122 of FIG. 7) if the patient's current weight ever exceeds 102 kilograms or drops below 98 kilograms (2% of 100 kilograms=2 kilograms).

It will be understood that, regardless of whether the user selects an adaptive threshold or a non-adaptive threshold via selector 134 (FIG. 9), controller 70 is configured to use the selected threshold (adaptive or non-adaptive) in its comparisons of the patient's current weight to the patient's initial weight reading 90a. Further, as noted, controller 70 issues a notification—such as the notification of changing the color, size, and/or configuration of indicator 120 (e.g. adding arrow 122)—whenever the patient's current weight reading changes by more than the selected threshold.

When the user selects an adaptive threshold via selector 134, controller 70 is configured to utilize an adaptive threshold for its comparisons between the patient's current weight and his or her initial weight reading 90a. The adaptive threshold may be calculated by controller 70 in a variety of different manners, depending upon the particular embodiment of patient support apparatus 20. In some embodiments, controller 70 is configured to take automatic patient weight readings over a time period and determine the mean absolute deviation of the patient weight readings taken over that time period. Once the mean absolute deviation is calculated, controller 70 selects a weight value based on that mean absolute deviation and uses that selected weight value as the threshold. For example, suppose that controller 70 calculates that the mean absolute deviation of the fluctuations in a patient's weight over a given time period is equal to 1.0 kilograms. Once this is calculated, controller 70 is configured to select a threshold value that is based on this mean absolute deviation, such as by multiplying the mean absolute deviation by a scale factor. In this particular example, with the mean absolute deviation of 1.0 kilogram, controller 70 might be configured to multiply it by 1.5 to yield a value of 1.5 kilograms to use as the threshold. In general, in order to avoid providing an excessive number of cumulative weight change notifications, controller 70 is configured to select a value for the threshold that is higher than the mean absolute deviation of the weight fluctuations.

The time period over which the fluctuations in the patient's weight are analyzed by controller 70 may vary. In at least one embodiment, controller 70 is configured to take repetitive readings of the patient's weight over a period of about six to twenty-four hours (although other time periods may be used), typically starting at the moment the patient first enters patient support apparatus 20 (which may be before the first patient weight reading 90a is recorded). The patient weight readings that are taken over this time period may be made at a variety of different frequencies. In one example, controller 70 is configured to automatically take patient weight readings every minute throughout this time period. More or less frequent weight readings may, or course, be implemented.

It will be understood that the weight readings taken by controller 70 over the time period are taken automatically by controller 70 without requiring any user intervention. That is, unlike the patient weight readings 90 that are stored in memory and that require a caregiver to activate the save weight control 114, the patient weight readings that are taken in order to determine an adaptive weight threshold are done without requiring the caregiver to take any weight readings using control 114, or any other controls on patient support apparatus 20 (other than using selector 134 to select the adaptive threshold). These automatically taken weight readings, in some embodiments, are not displayed to the user, but instead are used internally by controller 70 to calculate the mean absolute deviation, or another statistical parameter. Thus, these automatically-taken weight calculations will not show up on display 60 when a user presses on the weight history control 116 (see FIG. 8). However, in other embodiments, one or more of these automatically-taken weight readings may be added to this weight history and displayed in response to the activation of weight history control 116.

In some embodiments, controller 70 is adapted to allow a user to choose one or more characteristics of the threshold based on the statistical analysis it carries out on the patient's weight fluctuations. For example, in some embodiments, the user is able to select how much over or under the mean absolute deviation the threshold should be set. In some such embodiments, the user may be able to specify a particular value over or under the mean absolute deviation to use as the threshold (e.g. 1.0 kg above the mean absolute deviation). Alternatively, or additionally, controller 70 may provide one or more controls on display 60 that allow the user to specify a percentage (or other scale factor) of the mean absolute deviation that should be used as the threshold (e.g. set the threshold to be equal to 140% of the mean absolute deviation). Controller 70 may be further configured to allow the user to input other information regarding how the statistical analysis is to be used in setting the threshold.

In some embodiments, controller 70 is configured to automatically use a non-adaptive threshold during the time period when controller 70 is gathering statistics of the patient's weight fluctuations. Thus, for example, controller 70 may use a non-adaptive threshold (e.g. 2.0 kilograms) for the first eight to twenty-four hours, or so, that a patient is positioned on a patient support apparatus 20. Once the time period has expired, controller 70 performs the statistical analysis of the patient weight readings taken during that time period, calculates the adaptive threshold based on that statistical analysis, and then automatically switches from using the non-adaptive threshold to the adaptive threshold for the remainder of the patient's stay on the patient support apparatus. The non-adaptive threshold that is used during the time period when controller 70 is gathering statistical data about the patient's weight fluctuations may be a fixed value set by patient support apparatus 20, or it may be a value that the user can control (e.g. using slider bar 138).

In some embodiments, controller 70 is configured to allow the patient to choose the time period during which controller 70 takes readings of the patient's weight fluctuations. In such embodiments, controller 70 may display a slider similar to that shown in FIG. 9 in which the user can select the number of hours that controller 70 will gather statistical data for before calculating a statistical analysis of the patient's weight fluctuations. Once the time period is reached, controller 70 performs its statistical analysis of the patient's weight readings, determines the adaptive threshold, and then automatically switches from using the non-adaptive threshold to the adaptive threshold for the remainder of the patient's stay on the patient support apparatus 20.

Once the time period expires, in some embodiments, controller 70 is configured to stop taking automatic weight measurements. In other embodiments, controller 70 may be configured to continue to take automatic measurements of the patient's weight, and to use the automatic measurements to periodically update the results of the statistical analysis it previously performed, and then, as appropriate, update the adaptive threshold. Thus, suppose, for example, that controller 70 determines after seven hours of automatic weight measurements that the patient's mean absolute weight deviation is 1.5 kg, and controller 70 is configured to select an adaptive threshold of 2.0 kg as a result of that calculation. Controller 70 would then switch to using the adaptive threshold of 2.0 kg in its comparisons of the patient's current weight to the initial weight reading 90a. However, in some embodiments, controller 70 might continue to take additional automatic weight measurements such that at, say, eight hours, it would recalculate the mean absolute deviation of the patient's weight fluctuations and adjust the threshold, as appropriate. It can therefore be seen that, in some embodiments, the time period during which controller 70 gathers statistical data about the fluctuations in the patient's weight may extend beyond an initial set time period. Indeed, in some embodiments, controller 70 may be configured to perform statistical analyses of the patient's weight during the patient's entire stay on patient support apparatus 20.

In some embodiments, controller 70 is configured to use both an adaptive threshold and a non-adaptive threshold in combination with each other. For example, in some such embodiments, controller 70 is configured to compare the difference between the patient's current weight and the patient's initial weight 90a to both an adaptive and a non-adaptive threshold. If the difference exceeds either one of these thresholds, controller 70 is configured to issue a notification to the user (e.g. add arrow 122 to screen 100 FIG. 7). In such embodiments, controller 70 may be configured to adjust the adaptive threshold in a manner that is based on the amount of time of that controller 70 has been able to gather automatic measurements of the patient's weight. In other words, the adaptive threshold may be adjusted automatically based on how much data controller 70 has at its disposal for statistically analyzing.

It will be understood that, although the statistical analysis performed by controller 70 on the automatic patient weight readings has been primarily described herein as a mean absolute deviation analysis, controller 70 may be configured to perform other types of statistical analyses on the automatically gathered patient weight readings. As but one example, controller 70 may be configured to determine the standard deviation of the automatically gathered patient weight readings, and to then select a threshold that is based on the standard deviation (e.g. a threshold that is 1.5 standard deviations).

It will also be understood that, although the discussion herein has focused on using the same threshold (whether adaptive or non-adaptive) for both cumulative patient weight gains and cumulative patient weight losses, it will be understood that controller 70 could be configured to select—or to allow the user to select—different thresholds for gains and for losses in the patient's weight. For example, controller 70 is configured in some embodiments to use a first threshold for gains in the patient's weight, and to use a second and different threshold for losses in the patient's weight. If either of these thresholds are exceeded, then controller 70 issues a notification to the user.

In addition to, or in lieu of, the notifications provided on display 60 by controller 70 of cumulative patient weight changes that exceed the threshold, controller 70 is configured in some embodiments to send a message to patient support apparatus 20 whenever a patient's cumulative weight change exceeds the threshold. Controller 70 sends this message via transceiver 68. Patient support apparatus server 82, in some embodiments, is configured to forward this information to one or more portable electronic devices 86 that are associated with the caregiver(s) assigned to the particular patient in that particular patient support apparatus 20. In some embodiments, the portable electronic devices 86 are configured to display one or more screens that are the same as, or similar to, the screens 100, 100a, and 130 shown in FIGS. 6-9. Still further, in some embodiments, the portable electronic device 86 is configured to send one or more commands to patient support apparatus server 82 that get forwarded to patient support apparatus 20. The commands may instruct controller 70 to perform any of the actions that the user is able to take on any of the screens of FIGS. 6-9. Thus, a remotely positioned caregiver can, for instance, use his or her portable electronic device 86 to select a threshold using slider bar 138 of FIG. 9, or to save a patient weight using save weight control 114 of FIG. 7. Controller 70 may be configured to carry out still other commands from one or more portable electronic devices 86.

Controller 70 is configured, in at least some embodiments, to automatically remove the effects of objects added to, or removed from, patient support apparatus 20 when it is determining an adaptive threshold, as well as when it is comparing a patient's current weight reading to the patient's initial weight reading. In such embodiments, controller 70 automatically detects when an object is added or removed from patient support apparatus 20, enters or deletes the weight of the object in its weight log, and adjusts the weight readings from the force sensors 54 in a manner that removes the object's weight from the calculations and comparisons described herein. In some embodiments, this automatic detection of the addition and/or removal of objects from patient support apparatus 20 is carried out in any of the manners disclosed in the following commonly assigned U.S. patent references: U.S. patent application Ser. No. 63/255,211 filed Oct. 13, 2021, by inventors Sujay Sukumaran et al. and entitled PATIENT SUPPORT APPPARATUS WITH AUTOMATIC SCALE FUNCTIONALITY; U.S. Pat. No. 10,357,185 issued to Marko Kostic et al. on Jul. 23, 2019, and entitled PERSON SUPPORT APPARATUSES WITH MOTION MONITORING; U.S. Pat. No. 11,033,233 issued to Michael Hayes et al. on Jun. 15, 2021, and entitled PATIENT SUPPORT APPARATUS WITH PATIENT INFORMATION SENSORS; and U.S. patent application Ser. No. 16/992,515 filed Aug. 13, 2020, by inventors Kurosh Nahavandi et al. and entitled PATIENT SUPPORT APPARATUS WITH EQUIPMENT WEIGHT LOG, the complete disclosures of all of which have already been incorporated herein by reference.

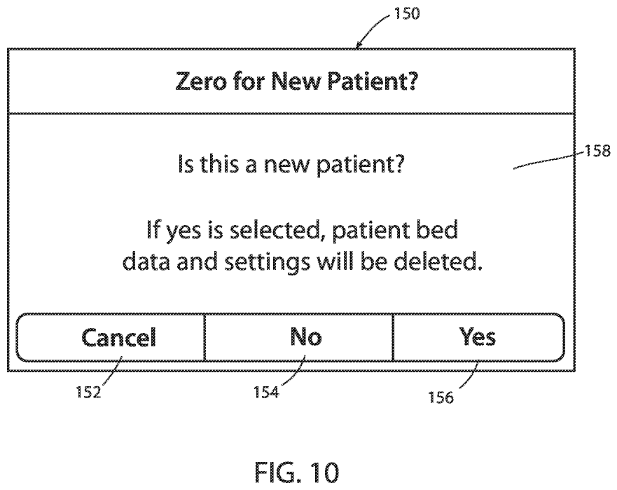

FIG. 10 illustrates an example of a new patient control screen 150 that controller 70 may display on display 60 in response to a user selecting the zero control 112 (e.g. FIGS. 6-8), and/or in response to other triggers (e.g. a user using other controls to navigate to screen 150). New patient control screen 150 includes a cancel control 152, a no control 154, and a yes control 156. Screen 150 also includes a message area 158 in which is displayed a message asking the caregiver to confirm that the patient assigned to patient support apparatus 20 is a new patient. If the user presses cancel control 152, controller 70 neither zeros the scale system nor concludes that a new patient has been assigned to patient support apparatus 20. If the user presses the no control 154, controller 70 proceeds to zero the scale system, but does not conclude that a new patient has been assigned to the patient support apparatus. If the user presses the yes control 156, controller 70 proceeds to both zero the scale and conclude that a new patient has been assigned to patient support apparatus 20.

When a caregiver informs controller 70 that a new patient has been assigned to patient support apparatus 20, controller 70 is configured to erase the saved patient weight readings 90 from memory 72. If controller 70 was previously using an adaptive threshold, controller 70 also erases the statistical analysis (including the data used in the statistical analysis) from memory 72. Thus, when a new patient is assigned to patient support apparatus 20, first patient weight reading 90a is set to zero (or is deemed empty) until the caregiver first uses the scale system to record a weight for that patient (e.g. using control 114 of FIG. 6). All of the other patient weight readings 90 are also set to zero until they are populated by patient weight readings that are made for that specific patient. Similarly, in some embodiments, to the extent patient support apparatus 20 had gathered statistical data regarding the previous patient's weight readings, those too are erased from memory 72 and controller 70 must take new automatic patient weight readings to calculate a new adaptive threshold for the new patient. The new automatic patient weight readings are performed in the manner previously described, and the statistical analysis of these readings may be performed in any of the same manners previously described. In an alternative embodiment, controller 70 may be configured to retain the statistical analysis of each patient's weight fluctuations in memory and to utilize that information in automatically selecting the threshold for a particular patient.

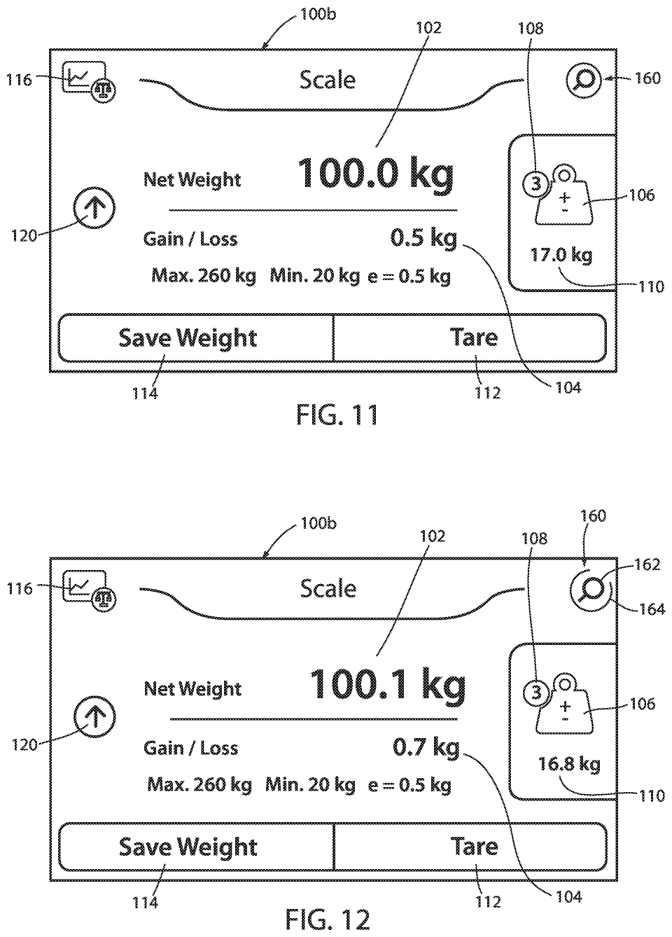

FIG. 11 illustrates another example of a scale screen 100b that may be displayed by controller 70 on display 60 in response to a user pressing on scale control 58c, and/or in response to other actions undertaken by the user. Scale screen 100b, like scale screen 100 (FIGS. 6-7), includes a number of items that are the same as found in scale screen 100, that operate in the same manner as was previously described, and that have the same reference number as found in scale screen 100. Such items include patient weight indicator 102, gain/loss indicator 104, weight log icon 106, object number 108, object total weight indicator 110, zero control 112, save weight control 114, and scale history control 116. These items are not described further herein.

Scale screen 100b differs from scale screen 100 in that it includes a high precision control 160. High precision control 160, when pressed, causes controller 70 to temporarily display weight readings with a higher level of precision than it normally displays by default. Thus, for example, in the example shown in FIG. 11, controller 70 is displaying the patient's current weight as 100.0 kg, as shown by patient weight indicator 102. Although not visible in FIG. 11, controller 70 is rounding the current patient's weight to the nearest half of a kilogram. The same is true for the weight that the patient has lost or gained (gain/loss indicator 104) and for the total weight of the items in the equipment log (total weight indicator 110). When the user presses on high precision control 160, controller 70 temporarily displays these weights with a higher level of precision. An example of this is shown in FIG. 12.

FIG. 12 illustrates scale screen 100b moments after the user has pressed on the high precision control 160 of FIG. 11. As can be seen therein, controller 70 has changed the patient's weight from being rounded to the nearest half of a kilogram (100.0 kg, as shown in FIG. 11 by patient weight indicator 102) to being rounded to the nearest 0.1 kilograms (100.1 kg, as shown in FIG. 12 by patient weight indicator 102). Similarly, controller 70 has changed the patient's weight loss indicator 104 from being rounded to the nearest half of a kilogram (0.5 kg, as shown in FIG. 11) to being rounded to the nearest 0.1 kilogram (0.7 kg, as shown in FIG. 12). Controller 70 has also changed the total weight indicator 110 from being rounded to the nearest half of a kilogram (17.0 kg, as shown in FIG. 11) to being rounded to the nearest 0.1 kilogram (16.8 kg, as shown in FIG. 12).

Controller 70 is configured to display the higher precision weight readings shown in FIG. 12 for a temporary amount of time. In some embodiments, this temporary amount of time is a fixed amount of time that starts at the moment the user presses on high precision control 160. For example, in some embodiments, controller 70 is configured to display higher precision weight readings, such as those shown in FIG. 12, for a time period of approximately ten seconds following the pressing of high precision control 160. After the expiration of this ten second time period, controller 70 automatically reverts to displaying the lower precision weight readings (e.g. what is shown in FIG. 11). It will be understood, of course, that the temporary time period can vary widely from the ten second example mentioned herein.

In some embodiments, controller 70 is configured to change the manner in which high precision control 160 is displayed on display 60 after the user presses on it. For example, as shown from a comparison of FIG. 11 to FIG. 12, controller 70 is configured to display control 160 with a dark magnifying glass icon 162 having a solid white background when the user has not recently pressed on control 160 (FIG. 11). After the user presses on control 160, controller 70 is configured to change the dark magnifying glass icon 162 to a white, or lighter colored, icon having a solid black (or dark) background. In addition, controller 70, in some embodiments, is further configured to display a circle icon 164 around the magnifying glass icon 162, wherein the amount of the circle icon 164 that is shown varies in accordance with the time remaining for the display of the high precision weight readings.

In other words, in some embodiments, controller 70 is configured to display circle icon 164 as a full circle when a caregiver first presses control 160, and to thereafter reduce the amount of circle icon 164 that is displayed. As a result, smaller and smaller portions of the circle icon 164 are displayed until, eventually, circle icon 164 is not displayed at all. At that moment, controller 70 is configured to revert back to displaying the lower precision weight readings (e.g. FIG. 11). Controller 70 is therefore configured to reduce the amount of the circle icon 164 that is displayed at a rate that matches the time remaining for the display of the high precision weight values. Thus, for example, if the high precision weight values are automatically displayed for ten seconds after the control 160 is pressed, controller 70 is configured to reduce the portion of circle icon 164 at a rate such that it completely disappears after ten seconds. In this manner, the varying portion of circle icon 164 that is displayed provides a visual indication of the amount of time remaining that the high precision values of FIG. 12 will continue to be displayed. The user needs only look at the portion of the circle icon 164 that is currently being displayed to get an idea of the amount of time remaining until controller 70 stops displaying the high precision weight values and instead switches to displaying the lower precision weight values.

In some embodiments, controller 70 is adapted to allow a user to manually deactivate the display of the high precision weight values (FIG. 12) prior to the time period expiring. This manual deactivation can be accomplished by the user pressing on high precision control 160 a second time. High precision control 160 can therefore act like a toggle switch for switching between higher and lower precision weight values. Controller 70 is also configured to stop displaying the higher precision weight values whenever the user navigates away from screen 100b and/or whenever display 60 goes to sleep.

Controller 70 is configured to display the lower precision weight values of FIG. 11 by default. That is, unless the caregiver presses on control 160, control 70 will always display lower precision weight values on screen 100b (and will only temporarily display higher precision weight values after control 160 is activated).

As can be seen in FIG. 12, the save weight control 114 and zero control 112 are both grayed out and non-functional. Controller 70 is configured to temporarily disable these controls 112 and 114 in response to the user pressing on control 160. Consequently, the user cannot save a patient's weight and/or tare the scale system when the high precision weight values are being used. Instead, the user must either wait until controller 70 stops displaying the high precision weight values, or the user manually terminates the display of the high precision weight values (e.g. by pressing control 160 a second time). Once controller 70 reverts back to displaying the lower precision weight values, it automatically re-enables zero control 112 and save weight control 114 so that the user is then able to use these controls.

It will also be understood that controller 70 may be configured to also monitor the outputs of force sensors 54 for implementing a patient exit detection system that may be armed and disarmed by a caregiver. When armed, the exit detection system is configured to issue an alert when the patient exits patient support apparatus 20, or moves beyond one or more boundaries. In some embodiments, the exit detection functions may be carried out in any off the manners disclosed in the following commonly assigned U.S. patent applications: Ser. No. 62/889,254 filed Aug. 20, 2019, by inventors Sujay Sukumaran et al. and entitled PERSON SUPPORT APPARATUS WITH ADJUSTABLE EXIT DETECTION ZONES, and/or Ser. No. 15/266,575 filed Sep. 15, 2016, by inventors Anuj K. Sidhu et al. and entitled PERSON SUPPORT APPARATUSES WITH EXIT DETECTION SYSTEMS, the complete disclosures of both of which are incorporated herein by reference.

It will also be understood that controller 70 may be configured to adjust the outputs of force sensors 54 to take into account the tilting of litter frame 28. That is, in some embodiments, force sensors 54 are load sensors whose outputs do not reflect the true load placed thereon when the load applied to the load cell is tilted, such as may happen when litter frame 28 is tilted out of a horizontal orientation. In such cases, the level of tilt is detected by one or more sensors onboard patient support apparatus 20 and a simple trigonometric calculation (based on the detected tilt angle) is applied to the outputs of the load cells 54 by controller 70 to remove this error in the load measurement. These tilt-adjusted load cell readings are then processed and used to compute more accurate readings of the patient's weight.

Various additional alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus comprising:
a support surface adapted to support a patient thereon;
a plurality of force sensors adapted to detect downward forces exerted on the support surface;
a control adapted to record a patient weight when activated by a caregiver;
a memory;
a display; and
a controller adapted to record in the memory an initial patient weight corresponding to a first time the caregiver uses the control to record a particular patient's weight, the controller further adapted to thereafter automatically compare a current patient weight to the initial patient weight and to provide a notification to the caregiver if the current patient weight differs from the initial patient weight by more than a threshold, the controller still further adapted to display a threshold selection screen on the display, the threshold selection screen adapted to allow the caregiver to adjust the threshold.

2. The patient support apparatus of claim 1 further comprising a new patient control adapted to be activated by the caregiver when a new patient is assigned to the patient support apparatus, wherein the controller is further adapted to erase the initial patient weight of the particular patient when the new patient control is activated.

3. The patient support apparatus of claim 2 wherein the controller is further adapted to store in the memory a new initial patient weight corresponding to a first time the caregiver uses the control to record a new patient's weight, and wherein the controller is further adapted to thereafter automatically compare a current weight of the new patient to the new initial patient weight and to provide the notification to the caregiver if the current weight of the new patient differs from the new initial patient weight by more than the threshold.

4. The patient support apparatus of claim 1 wherein the controller is further adapted to display a patient weight gain/loss indicator on the display, the patient weight gain/loss indicator providing an indication to the caregiver of a difference between the current patient weight and a last saved patient weight, wherein the last saved patient weight corresponds to a weight of the patient recorded when the caregiver most recently activated the control.

5. The patient support apparatus of claim 1 further comprising a network transceiver adapted to communicate with a server hosted on a healthcare facility computer network, and wherein the controller is further configured to send a notification message to the server if the current patient weight differs from the initial patient weight by more than the threshold.

6. The patient support apparatus of claim 1 wherein the controller is further adapted to display an adaptive threshold option on the display, wherein if the caregiver selects the adaptive threshold option, the controller is further adapted to switch to providing the notification to the caregiver if the current patient weight differs from the initial patient weight by more than an adaptive threshold.

7. The patient support apparatus of claim 6 wherein the controller is adapted to select a value for the adaptive threshold that is based on fluctuations in the patient's weight over a time period.

8. The patient support apparatus of claim 7 wherein the controller is further adapted to determine a mean absolute deviation of the fluctuations and to select the value for the adaptive threshold based on the mean absolute deviation of the fluctuations.

9. The patient support apparatus of claim 8 wherein the controller is further adapted to select the value for the adaptive threshold that is greater than the mean absolute deviation.

10. The patient support apparatus of claim 7 wherein the controller is further adapted to increase the time period.

11. The patient support apparatus of claim 1 wherein the threshold selection screen includes a slider icon displayed thereon adapted to be repositioned by the caregiver, and wherein the caregiver adjusts the threshold by sliding the slider icon.

12. The patient support apparatus of claim 11 wherein the slider icon is adapted to slide within a range of values for the threshold, and wherein the range includes values between at least one and two kilograms.

13. A patient support apparatus comprising:
a support surface adapted to support a patient thereon;
a plurality of force sensors adapted to detect downward forces exerted on the support surface;
a control adapted to record a patient weight when activated by a caregiver;
a memory;
a display; and
a controller adapted to record in the memory an initial patient weight corresponding to a first time the caregiver uses the control to record a particular patient's weight, the controller further adapted to thereafter automatically compare a current patient weight to the initial patient weight and to provide a notification to the caregiver if the current patient weight differs from the initial patient weight by more than an adaptive threshold, wherein the controller is adapted to select a value for the adaptive threshold that is based on fluctuations in the patient's weight over a time period.

14. The patient support apparatus of claim 13 wherein the controller is further adapted to determine a mean absolute deviation of the fluctuations and to select the value for the adaptive threshold based on the mean absolute deviation of the fluctuations.

15. The patient support apparatus of claim 14 wherein the controller is further adapted to select the value for the adaptive threshold that is greater than the mean absolute deviation.

16. The patient support apparatus of claim 13 wherein the controller is further adapted to increase the time period.

17. The patient support apparatus of claim 13 further comprising a new patient control adapted to be activated by the caregiver when a new patient is assigned to the patient support apparatus, wherein the controller is further adapted to erase the initial patient weight of the particular patient when the new patient control is activated.

18. The patient support apparatus of claim 17 wherein the controller is further adapted to store in the memory a new initial patient weight corresponding to a first time the caregiver uses the control to record a new patient's weight, and wherein the controller is further adapted to thereafter automatically compare a current weight of the new patient to the new initial patient weight and to provide the notification to the caregiver if the current weight of the new patient differs from the new initial patient weight by more than the adaptive threshold.

19. The patient support apparatus of claim 13 wherein the controller is further adapted to display a patient weight gain/loss indicator on the display, the patient weight gain/loss indicator providing an indication to the caregiver of a difference between the current patient weight and a last saved patient weight, wherein the last saved patient weight corresponds to a weight of the patient recorded when the caregiver most recently activated the control.

20. The patient support apparatus of claim 13 further comprising a network transceiver adapted to communicate with a server hosted on a healthcare facility computer network, and wherein the controller is further configured to send a notification message to the server if the current patient weight differs from the initial patient weight by more than the adaptive threshold.

21.-64. (canceled)
